(19) United States
(12) Patent Application Publication
(10) Pub. No.: US 2013/0130910 A1
(43) Pub. Date: May 23, 2013

(54) SUSTAINED RELEASE MICROPARTICLES AND SUSTAINED RELEASE MICROPARTICLE-CONTAINING PREPARATIONS

(71) Applicants: Youichi Hori, Nagano-shi (JP); Satoru Ishimori, Nagano-shi (JP); Mitsuyoshi Sato, Nagano-shi (JP)

(72) Inventors: Youichi Hori, Nagano-shi (JP); Satoru Ishimori, Nagano-shi (JP); Mitsuyoshi Sato, Nagano-shi (JP)

(21) Appl. No.: 13/809,444

(22) PCT Filed: Jul. 15, 2011
(86) PCT No.: PCT/JP2011/066183

§ 371 Date: Feb. 5, 2013

(30) Foreign Application Priority Data
Jul. 15, 2010 (JP) 2010-160604

(51) Int. Cl. A01N 25/10; A23L 1/00; B01J 35/02; A61K 9/14; C12N 11/08

(52) U.S. Cl. CPC A01N 25/10; A61K 9/14; C12N 11/08; B01J 35/023; A23L 1/0044
USPC 504/360; 424/497; 435/182; 424/468; 502/159; 424/419; 426/89; 71/27; 512/4; 510/441; 523/218; 427/180

(57) ABSTRACT

[Problem]

Provision of sustained release microparticles having excellent characteristics, in which the microparticles for supporting a substance or substances can fully control the release of the supported substances, can sufficiently diffuse the supported substances in a medium such as water, and can effect sustained release of more than one kind of the supported substances differing in water solubility, each at the respectively desired rate; and also to provide preparations which contain said sustained release microparticles.

[Means of Solution]

Sustained release microparticles comprising a supported substance, microgranular form and coating agent, said microgranular form having an average particle size within a range of from 20 to 300 μm; and preparations which contain the sustained release microparticles.

### TECHNICAL FIELD

This invention relates to sustained release microparticles having a controlled outward release rate of the substance which is supported thereon and preparations containing the sustained release microparticles.

### BACKGROUND ART

It has been heretofore practiced in the art of sustained release preparations, to have various bases support such materials as coloring matter, flavor and fragrance, agrochemical, drug, fertilizer, enzyme, physiologically active substance, exothermic substance, endothermic substance, antistatic agent, rust inhibitor, and the like, and to have them effect sustained release of the supported substances.

Proposals made in the past include, for example, porous microparticles containing therein coloring matter, flavor and fragrance, agrochemical or the like, with their surfaces coated with a specific compound (Patent Document 1, Patent Document 2); petal-like porous base with calcium carbonate serving as the nucleus material, having a petal-like porous structure and specific physical properties (Patent Document 3); sustained release metal oxide hollow microparticles of porous and hollow structure containing a liquid substance therein (Patent Document 4); and inorganic porous microparticles in which a functional material is enclosed (Patent Document 5).

However, it is difficult to fully control the release property of such a substance supported on a base or microparticles, with those conventional supporting type microparticles as above. Furthermore, these conventional base or microparticles having sustained release property are subject to such problems as, when the supported substance is slowly discharged into a medium such as water, occasionally it stays around the discharged site and fails to sufficiently diffuse; or it is difficult to have more than one kind of supported substances differing in water-solubility at respectively desired discharge rates.

### PRIOR ART DOCUMENTS

### Patent Documents

Patent Document 1: JP 2003-286196A
Patent Document 2: JP 2009-12996A
Patent Document 3: JP Hei 10 (1998)-182492A
Patent Document 4: JP Hei 6 (1994)-285358A
Patent Document 5: JP Hei 7 (1995)-173452A

### SUMMARY OF THE INVENTION

### The Problems to be Solved

The object of the invention is to provide sustained release microparticles having excellent characteristics, in which the microparticles for supporting a substance or substances can fully control the release of the supported substances, can sufficiently diffuse the supported substances in a medium such as water, and can effect sustained release of more than one kind of the supported substances differing in water solubility, each at the respectively desired rate; and also to provide preparations which contain said sustained release microparticles.

### Means of Solution

We engaged in concentrative studies with the view to solve the problems and have now come to discover that use of microgranular form having an average particle size within the range of from 20 to 300 μm, as the carrier for supporting the substances to be supported, provides sustained release microparticles whose release rate of the supported substance(s) is sufficiently controlled; that a preparation which comprises said sustained release microparticles is capable of sufficiently diffusing the supported substance into a medium such as water, not allowing it to stay around the site of its sustained release and, furthermore, is capable of releasing more than one kind of the supported substances, which differ from each other in water solubility, at respectively desired release rates in sustained manner. The present invention is whereupon completed.

Accordingly, therefore, the invention provides sustained release microparticles comprising a supported substance, microgranular form, and coating agent, which are characterized in that the microgranular form has an average particle size within the range of from 20 to 300 μm.

The invention also provides a preparation containing sustained release microparticles, which comprises at least one kind of the sustained release microparticles.

Hereinafter the sustained release microparticles and the preparation comprising the sustained release microparticles are explained in further details.

### EMBODIMENTS FOR WORKING THE INVENTION

### Supported Substance:

As the substances suitable to be supported on the sustained release microparticles of the invention, for example, included are agrochemically active component, coloring matter, flavor and fragrance, functional substance, drug, fertilizer component, enzyme, physiologically active substance and the like.

Agrochemically active component includes all of the active components which are generally used as the active ingredients of agrochemicals such as the substances for controlling noxious organisms in the fields of agriculture and horticulture, and substances for regulating plant or vegetable growth, for example, herbicide, plant growth regulating agent, insecticide, miticide, antimicrobial agent and antifungal agent.

Examples of herbicide include Pyraclonil, Propyrisulfuron, Benzobicyclon, Pyrazolate, Tefuryltrione, Mesotrione, Topramezone, Sulcotrione, Oxaziclomefone, Benzofenap, Pyrazoxyfen, Pyributicarb, Butamifos, Mefenacet, Bensulfuron-methyl, Anilofos, Butachlor, Pretilachlor, Thiobencarb, Chlornitrofen, Chlomethoxyfen, Dymron, Bifenox, Naproanilide, Oxadiazon, Oxadiargyl, Bentazon, Molinate, Piperophos, Dimepiperate, Esprocarb, Dithiopyr, Benfuresate, Quinoclamin, Cinmethylin, MCPA or salts thereof such as a sodium salt or potassium salt, or esters thereof; 2,4-D or salts thereof such as a sodium salt or potassium salt, or esters thereof; MCPB or salts thereof such as a sodium salt or potassium salt, or esters thereof; Quinclorac, Pyrazosulfuron-ethyl, Pentoxazone, Thenylchlor, Indanofan, Cumyluron, Clomeprop, Cinosulfuron, Simetryn, Dimethametryn, Cyhalofop-butyl, Etobenzanide, Cafenstrole, Ethoxysulfuron, Azimsulfuron, Cyclosulfamuron, Imazosulfuron, Flucetosulfuron, Orthosulfamuron, Halosulfuron-methyl, Bispyribac, Bispyribac-sodium, Pyriminobac-methyl, Pyriftalid, Fentrazamide, Ipfencarbazone, Metazosulfuron, Pyrimisulfan, Penoxsulam and Fenoxasulfone.

Examples of plant growth regulating agent include Inabenfide, Mepiquat chloride, Chlormequat, Flurprimidol, Bispyribac-sodium, Paclobutrazol, Prohexadione-calcium, Trinexapac-ethyl, Daminozide, Uniconazole P and Triapenthenol.

Examples of insecticide and miticide include Isoxathion, Diazinon, Disulfoton, Malathon, Propaphos, Trichlorfon, Formothion, Dimethoate, Monocrotophos, Acephate, Carbofuran, Carbosulfan, Thiocyclam, Cartap, Bensultap, Benfuracarb, Furathiocarb, Buprofezin, Fenobucarb, Metolcarb, Propoxur, Imidacloprid, Nitenpyram, Acetamiprid, Chlorpyrifos-methyl, Dimethylvinphos, Pyridaphenthion, Thiodicarb, Dinotefuran, Clothianidin, Thiacloprid, Thiamethoxam, Chromafenozide, Halofenozide, Tebufenozide, Methoxyfenozide, Fipronil, Ethiprole, Nicotine sulfate, Spinosad, Pymetrozine, Flonicamid, Chlorantraniliprole, Cyantraniliprole, Spinetoram, Cycloprothrin, Etofenprox, Silafluofen, Flubendiamide and Pyrifluquinazon.

Examples of antimicrobial agent and antifungal agent include Probenazole, Isoprothiolane, Iprobenfos, Tricyclazole, Pyroquilon, Carpropamide, Azoxystrobin, Flutolanil, Mepronil, Thifluzamide, Furametpyr, Teclofthalam, Benomyl, Diclocymet, Fenoxanil, Phthalide, Metominostrobin, Orysastrobin, Kasugamycin, Validamycin, Oxytetracycline, Streptomycin, Ferimzone, Simeconazole, Copper, Thiophanate-methyl, EDDP, IBP, Metalaxyl, Iprodione, Hydroxyisoxazole, Oxolinic acid, Penthiopyrad, Diclomezine, Tiadinil, Acibenzolar-S-methyl and Isotianil.

Examples of the coloring matter include various dyestuffs, pigments and synthetic dyes; those of the flavor and fragrance include essential oils extracted from plants and synthetic perfume; those of functional substance include catalysts for controlling reactions, color-developing agents to be used in combination with dyes, decolorizer, flame-retarding agent, defoaming agent and antistatic agent; those of drug include antibacterial agent and disinfectant; those of fertilizing component include nitrogen, phosphoric acid, potassium, magnesium and calcium carbonate; those of enzyme include diastase, protease, cellulase and lipase; and those of physiologically active substance include water-soluble vitamins such as vitamin B1, vitamin B2, vitamin B6, vitamin B12 and vitamin C; fat-soluble vitamins such as vitamin A, vitamin D and vitamin E; and amino acids such as lysine and glutamic acid.

These substances to be supported can be used either singly or in combination of two or more, and the content of the supported substance(s) in the sustained release microparticles can be within a range of, based on the weight of the sustained release microparticles, generally from 0.1 to 95 wt %, preferably from 0.5 to 90 wt %, inter alia, from 1 to 80 wt %.

### Microgranular Form

The microgranular form useful for the sustained release microparticles of the present invention can have either hollow or non-hollow structure.

As hollow microgranular form, microgranules having one or plural independent air bubbles are useful, examples of which include hollow glass microspheres made of sirasu obtained by calcining processing of volcanic glassy powder which is occasionally referred to as foamed sirasu (sirasu balloon); foamed pearlite made of pearlite or obsidian; foamed glass obtained by adding a foaming agent to glass powder and melting, foaming and calcining the same; fly ash obtained by using coal as a fuel; and hollow microballoons made of copolymers having acrylonitrile or methacrylic acid ester as a monomer component or phenolic resins.

As the non-hollow microgranular form, microgranules which can have open porous structure can be used, examples of which include active carbon, silica gel, alumina gel, ceramic powder obtained by calcining metal oxide or hydroxyapatite, diatomaceous earth, zeolite, white carbon and sand.

Of those microgranular forms useful for the sustained release microparticles of the invention, hollow microgranular forms, in particular, hollow glass microspheres, are preferred.

Hollow glass microspheres are those occasionally called foamed sirasu (sirasu balloon) which are hollow microspherical forms obtained from, for example, volcanic glassy powder as the starting material through such steps as heating to reduce the water content at the powder surface to not higher than 0.3%, immediately feeding the same into an internal-combustion type medium-fluidized bed oven together with an air current and fuel, and foaming the product by heating at about 900° to about 1,200° C. In the occasion of said foaming, hollow glass microspheres of more stable and uniform particle size can be obtained by controlling the water content inside the volcanic glassy powder to a range from 4.0 to 6.0%, in particular, from 4.1 to 5.8%.

Here the water content at the surface of volcanic glassy powder can be known from the difference in weight of the sample volcanic glassy powder between that measured immediately after its heating at 130° C. for 10 minutes and that before the heating. Whereas, the internal water content of the volcanic glassy powder can be determined from the difference in weight of the volcanic glassy powder between that measured immediately after its heating at 250° C. for 10 minutes and that measured immediately after its heating at 130° C. for 10 minutes.

The microgranular form useful for the present invention has an average particle size ranging generally from 20 to 300 μm, preferably from 20 to 150 μm, inter alia, from 20 to 80 μm; a floating ratio on water ranging generally from 20 to 95%, in particular, from 30 to 80%; and a water absorption generally ranging from 10 to 170%, in particular, from 20 to 130%.

In the present invention, the “average particle size” is a value measured with a laser diffraction particle size distribution analyzer. “Floating ratio on water” is the ratio of the sample floating on water to the total sample. More specifically, it is calculated through the following steps: put 100 ml of water and 5 g of the sample in a separating funnel, add further 150 ml of water, stir well, let the funnel stand for an hour, discharge the sediment and water, add 250 ml of water again to the remaining residue, repeat the above stirring, standing and discharging operations, recover the residue, dry and weigh the same, and calculate the ratio of the weight to 5 g of the sample.

The “water absorption” is measured as follows: set a funnel with a filter paper laid therein on a flask, thoroughly wet the filter paper with water under suction, measure the tare weight (the funnel+water-absorbed filter paper) at the time when the intervals of falling waterdrops become 3 seconds; separately, put 200 ml of refined water and 20 g of sample in a beaker, stir the content thoroughly, allow it to stand for 5 minutes, pour the refined water-containing mixture in the beaker into the funnel, measure the total weight after the water absorption (the funnel+water-absorbed filter paper+sample) at the time when the intervals of falling waterdrops become 3 seconds; and calculate the water absorption according to the following equation:

$$water absorption ( \% ) = \frac{total weight after water adsorption ( g ) - tare weight ( g ) + 20}{20} \times 100$$

Specific examples of the hollow glass microspheres include MAARLITE 713D, MAARLITE 722B, MAARLITE 732C, MAALITE 735B and MAARLITE BA-15 (tradenames, Marunaka-Hakudo Co., Ltd.); TOWANALITE SYB-5000, TOWANALITE SYB-2000, TOWANALITE SYB-1000H, TOWANALITE SYB-1000S and TOWANALITE SYB-0005 (tradenames, Towana K.K.) DAISETSU BALLOON-B (tradename, Bieihakudo Ind. Co. Ltd.); and SIRASU BALLOON (tradename, NeoLite Kohsan K.K.). Specific examples of foamed pearlite include HARDLITE B-03, HARDLITE B-04 and HARDLITE B-05 (tradenames, Showa Chemical Industry Co., Ltd.). Specific examples of foamed glass include LIGHTIC GL-F (tradename, Keiwa Rozai Co., Ltd.). As the fly ash, for example, KAINOSPHERES 150, KAINOSPHERES 300 and KAINOSPHERES 600 (tradenames, Taiheiyo Cement Corp.) can be used. As the hollow microballoon, for example, MICROSPHERE-F-30E and MICROSPHERE-F-80E (tradenames, Matsumoto Yushi-Seiyaku Co., Ltd.) and BJO-0930CB (tradename, Tomoe Kogyo Co., Ltd.) can be used.

These microgranular forms can be used either singly or in combination of two or more kinds.

The content of the microgranular form in the sustained release microparticles can be within a range of, based on the weight of the sustained release microparticles, generally from 0.1 to 90 wt %, preferably from 1 to 80 wt %, inter alia, from 2 to 70 wt %.

### Coating Agent:

The coating agent usable for the sustained release microparticles of the present invention is a substance which is useful for coating the microgranular form with the substance to be supported thereon. Any coating agent which can form a coating difficultly permeable for the supported substance can be used in general, without any particular limitation. For example, higher saturated fatty acid, wax, thermoplastic resin, thermosetting resin and the like can be used.

Examples of useful higher saturated fatty acid include stearic acid, zinc stearate, stearic acid amide and ethylenebis-stearic acid amide; those of wax include synthetic waxes such as polyethylene wax, carbon wax, Hoechst wax, and fatty acid ester; natural waxes such as carnauba wax, bees wax and Japan wax; and petroleum waxes such as paraffin wax and petrolatum. Examples of thermoplastic resin include polyolefins such as polyethylene, polypropylene, polybutene and polystyrene; vinyl polymers such as polyvinyl acetate, polyvinyl chloride, polyvinylidene chloride, polyacrylic acid, polymethacrylic acid, polyacrylate and polymethacrylate; diene polymers such as butadiene polymer, isoprene polymer, chloroprene polymer, butadiene-styrene copolymer, ethylene-propylene-diene copolymer, styrene-isoprene copolymer, MMA-butadiene copolymer and acrylonitrile-butadiene copolymer; polyolefin copolymers such as ethylene-propylene copolymer, butene-ethylene copolymer, butene-propylene copolymer, ethylene-vinyl acetate copolymer, ethylene-acrylic acid copolymer, styrene-acrylic acid copolymer, ethylene-methacrylic acid copolymer, ethylene-methacrylic ester copolymer, ethylene-carbon monoxide copolymer, ethylene-vinyl acetate-carbon monoxide copolymer, ethylene-vinyl acetate-vinyl chloride copolymer and ethylene-vinyl acetate-acrylic copolymer; and vinyl chloride copolymers such as vinyl chloride-vinyl acetate copolymer and vinylidene chloride-vinyl chloride copolymer. Examples of thermosetting resin include polyurethane resin, epoxy resin, alkyd resin, unsaturated polyester resin, phenolic resin, urea-melamine resin, urea resin and silicone resin. Of those, thermoplastic acrylic ester resin, butadiene-styrene copolymer resin, thermosetting polyurethane resin and epoxy resin are preferred, and among the preferred resins, particularly thermosetting polyurethane resin is preferred.

These coating agents can be used either singly or in combination of two or more kinds.

The content of the coating agent in the sustained release microparticles can be within a range of, based on the weight of the sustained release microparticles, generally from 0.1 to 60 wt %, preferably from 2 to 50 wt %, inter alia, from 3 to 40 wt %.

### Other Additives

The sustained release microparticles of the present invention can comprise, other than aforesaid supported substance, microgranular form and coating agent, additives such as surfactant, carrier, and the like. Furthermore, in such a case that the supported substance is unstable, a stabilizer such as pH-regulating agent, antioxidant, photostabilizer, desiccant or the like may be incorporated, where necessary.

### Preparation of Sustained Release Microparticles

The sustained release microparticles of the present invention can be prepared, for example, by the methods as described hereunder, in which still other additives may be incorporated where necessary:

(1) a method comprising mixing the substance to be supported, microgranular form and coating agent together and drying the mixture;

(2) a method comprising mixing the substance to be supported with the microgranular form, adding further the coating agent, mixing and drying the mixture; or mixing the microgranular form with the coating agent, further adding thereto the substance to be supported, mixing and drying the mixture; or mixing the substance to be supported with the coating agent, further adding thereto the microgranular form, mixing and drying the mixture.

(3) a method comprising dissolving the substance to be supported in an organic solvent, mixing the solution with the microgranular form, distilling the organic solvent off, further mixing the remainder with the coating agent and drying the mixture; or dissolving the substance to be supported in an organic solvent, mixing the solution with the microgranular form and coating agent, distilling the organic solvent off and further drying the coating agent; and

(4) a method comprising mixing a solution or dispersion, which has been formed by dissolving or dispersing the substance to be supported in water and a water-soluble medium, with the microgranular form, drying the mixture, further mixing the coating agent therewith and drying the mixture; or mixing the solution or dispersion, which has been formed by dissolving or dispersing the substance to be supported in water and a water-soluble medium, with the microgranular form and the coating agent, and drying the mixture.

Here mixing of the starting materials can be performed in conventional manner, for example, by such methods as: mixing liquid substance(s) with solid substance(s) in a single vessel; mixing solid substance(s) with solid substance(s) in a single vessel; or by spraying liquid substance(s) onto solid substance(s) in a single vessel to effect their mixing.

Drying also can be conducted following conventional methods such as normal temperature drying or heat-drying. As the temperature in heat-drying, such that induces no denaturation or decomposition of the substance to be supported, e.g., a temperature within the range of from about 40 to 120° C., is preferred.

Distillation of organic solvent can be effected in conventional manner, such as vacuum distillation, distillation by heating or vacuum distillation under heating.

The organic solvent used for dissolving the substance to be supported is subject to no particular limitation, so long as it is stable in the occasion of distillation, examples of which include: aromatic or aliphatic hydrocarbons such as xylene, toluene, alkylnaphthalene, phenylxylylethane, kerosene, light oil, hexane and cyclohexane; halogenated hydrocarbons such as chlorobenzene, dichloromethane, dichloroethane and trichloroethane; alcohols such as methanol, ethanol, isopropyl alcohol, butanol, hexanol and ethylene glycol; ethers such as diethyl ether, ethylene glycol dimethyl ether, tetrahydrofuran and dioxane; esters such as ethyl acetate and butyl acetate; ketones such as acetone, methyl ethyl ketone, methyl isobutyl ketone and cyclohexanone; nitriles such as acetonitrile and isobutyronitrile; acid amides such as dimethylsulfoxide, N,N-dimethylformamide and N,N-dimethylacetamide; and vegetable oils such as soybean oil and cotton seed oil. Of these, dichloromethane, acetone and methanol are particularly preferred.

The water-soluble medium used in the occasion of dissolving or dispersing the substance to be supported is subject to no particular limitation so long as it is soluble or dispersible in water, examples of which include starch, dextrin, carboxymethyl cellulose or salts thereof, sodium alginate, ammonium alginate, gelatin, casein, polyethylene glycol, polyvinyl pyrrolidone and glycerin. Such a water-soluble medium can be used within the range of, per 100 wt parts of water, normally from 0.1 to 70 wt parts, preferably from 0.1 to 60 wt parts, inter alia, from 0.1 to 50 wt parts.

### Sustained Release Microparticle-Containing Preparations:

Sustained release microparticles of the present invention can be formulated into various forms of preparation, and the resulting sustained release microparticles-containing preparations are useful as, for example, agrochemicals (including noxious organism controlling agent), fertilizer, coating agent, detergent, aromatic, catalyst, controlling agent, antibacterial agent or the like, according to the kind of the substance supported or contained in the sustained release microparticles.

The preparation can contain at least one kind of the sustained release microparticles. For example, it is possible to have the preparation contain plural kinds of the sustained release microparticles supporting each desired substances as the active components, to cause it to exhibit the effects of the respective supported substances simultaneously.

The preparation containing at least one kind of the sustained release microparticles of the present invention is hereafter referred to as the sustained release microparticle-containing preparation, which can take various forms of preparation according to the intended utility, for example, granule, fine granule, tablet, wettable powder, water dispersible granule, dustable powder, suspension concentrate, emulsion water in oil, emulsion oil in water, soluble concentrate, emulsifiable concentrate or oil miscible liquid. Of these, granule and tablet are preferred. In the present specification, the sustained release microparticle-containing granular preparations are referred to as sustained release microparticles-containing granule.

The sustained release microparticle-containing preparation can exhibit, as a single preparation, plural effects attributable to plural kinds of the supported substances, when it contains at least two kinds of the sustained release microparticles. For example, when the single preparation is formulated to contain plural supported substances which differ in water solubility and of which elution is so designed that they are released at respectively desired elution rates, the sustained release microparticle-containing preparation can release the plural supported substances at respectively desired elution rates.

The sustained release microparticle-containing preparation according to the invention can comprise, besides the sustained release microparticles, various formulation additives such as surfactant, binder, carrier and the like, whereby it becomes possible to promote diffusion of the supported substance(s) in water as they are released from the sustained release microparticles, not allowing them to stay around the site of their release. Furthermore, when the supported substance(s) are unstable, the preparation may further contain, where necessary, a stabilizer such as pH-regulating agent, antioxidant, photostabilizer or desiccant.

The content of the sustained release microparticles in the sustained release microparticle-containing preparation of the invention varies substantially, depending on the kind of the supported substance(s) in the sustained release microparticles. In general terms, however, it can be within the range of, based on the weight of the sustained release microparticle-containing preparation, from 0.1 to 60 wt %, preferably from 0.5 to 50 wt %, inter alia, from 1 to 40 wt %.

Surfactant which can be used in the sustained release microparticle-containing preparation is subject to no particular limitation, so long as it is useful for such purposes as imparting wettability or collapsibility to the granular form, promoting suspension, dispersion or diffusion of the supported substance(s) in water, or improving granulation of powdered starting materials. Specific examples of the surfactant include anionic surfactants such as alkylbenzenesulfonate, alkylnaphthalenesulfonate, naphthalenesulfonate-formaldehyde condensate, dialkylsulfosuccinate, dialkylsulfosuccinate sodium salt, lignosulfonate, α-olefin sulfonate, alkanesulfonate, polystyrenesulfonate, alkyl ether sulfate salt, polyoxyethylenealkanediol, polyoxyethylene alkyl ether sulfate salt, polyoxyethylene alkyl phenyl ether sulfate salt, polyoxyethylene polystyryl phenyl ether sulfate salt, polyoxyethlene alkyl ether phosphate salt, polyoxyethylene alkyl phenyl ether phosphate salt, polyoxyethylene polystyryl phenyl ether phosphate salt, fatty acid salt, metal salt of polycarboxylic acid, diisobutylene-maleic acid copolymer salt, isobutylene-maleic acid copolymer salt, styrene-maleic acid copolymer salt, and polyacrylate; nonionic surfactants such as polyoxyethylene alkyl ether, polyoxyetheylene alkyl phenyl ether, polyoxyethylene stilphenyl ether, polyoxyethylene stilphenyl ether polymer, polyoxyethylene sorbitan alkyl ester, polyoxyethylene/polyoxypropylene block polymer, polyoxyethylene alkyl ester and polyoxyethylene alkylamine; nonionic anion type surfactants such as ammonium salt of polyoxyethylene allyl phenyl ether sulfate, sodium salt of polyoxyethylene allyl phenyl ether sulfate, ammonium salt of polyoxyalkylene allyl phenyl ether sulfate and sodium salt of polyoxyalkylene allyl phenyl ether sulfate; silicone surfactants; and fluorine-containing surfactants.

These surfactants can be used either alone or in combination of two or more. The blend ratio of the surfactant can be within the range of, based on the weight of the sustained release microparticle-containing preparation, generally from 0.1 to 30 wt %, preferably from 0.1 to 20 wt % inter alia, from 0.1 to 15 wt %.

It is sufficient for the binder, which can be used in the sustained release microparticle-containing preparation of the invention, to have an ability to impart sufficient coherence to the preparation in the occasion of formulating various powdery starting materials into the preparation form, and at the same time, to have the ability to adequately bind the supported substance(s) to the microglanular form, and any known binder for use in formulating preparations can be used without limitation.

Specific examples of such binder include starch, dextrin, cellulose, methyl cellulose, ethyl cellulose, carboxymethyl cellulose or salt thereof, hydroxyethyl cellulose, hydroxypropyl cellulose, carboxymethyl starch, pullulan, sodium alginate, ammonium alginate, propylene glycol alginate, guar gum, locust bean gum, gum arabi, xanthene gum, gelatin, casein, polyvinyl alcohol, polyethylene oxide, polyethylene glycol, ethylene/propylene block polymer, sodium polyacrylate and polyvinyl pyrrolidone.

These binder can be used either alone or in combination of two or more. The blend ratio of the binder can be within the range of, based on the weight of the sustained release microparticle-containing preparation, generally from 0.1 to 30 wt %, preferably from 1 to 20 wt %, inter alia, from 2 to 10 wt %.

Carriers which have been used for preparations in general can be used also in the sustained release microparticle-containing preparation of the present invention without any particular limitation. Specific examples include inorganic carriers such as clay, bentonite, talc, calcium carbonate, sodium carbonate, zeeklite, sericite, acidic terra abla, silica, diatomaceous earth, pumice, zeolite, vermiculite, white carbon, pearlite, and attapulgite; and organic carriers such as octenylsuccinate, starch glucose, maltose, sucrose and lactose. Examples of water-soluble carrier include sodium tripolyphosphate, potassium chloride, urea, ammonium sulfate, sodium sulfate, sodium hydrogencarbonate, glucose, sucrose, fructose and lactose, and examples of water-floatable carrier include minerals such as foamed pearlite, foamed pumice and calcined vermiculite; plant materials such as cork, wood flour, cellulose and kenaf; synthetic resin fine granules such as foamed polystyrol fine granule and vinyl chloride resin powder; and plastic hallow bodies such as microspheres.

These carriers can be used either alone or in combination of two or more. The blend ratio of the carrier based on the weight of the sustained release microparticles-containing preparation can be within the range of generally from 0.1 to 95 wt %, preferably from 1 to 90 wt %, inter alia, from 10 to 90 wt %.

The preparations of the invention in which the sustained release microparticles support agrochemically active component are useful for controlling noxious organisms inhabiting in trees, flowering plants, gardens, upland field, orchards or paddy fields, and for regulating plant growth. Here the noxious organisms include undesirable harmful organisms and annoying organisms, such as weeds, plants, insects, mites, arthropods, nematodes, bacteria, filamentous fungi and viruses.

The sustained release microparticle-containing granular preparation of the invention, i.e., sustained release microparticle-containing granule, can be prepared by processing the so far described components following the granulation method normally adopted in the art of formulating medicaments. As the granulation method, for example, extrusion granulation, tumbling granulation, tumbling and fluidized granulation, fluidized bed granulation, compression granulation, stirring and mixing granulation, coating granulation or tabletting can be used, extrusion granulation being generally preferred, which can be effected as in the following specific example.

Sustained release microparticle-containing granule can be obtained by mixing the sustained release microparticles, a surfactant, binder, carrier and a stabilizer, kneading the mixture under addition of water, and granulating it with an extrusion granulator with its extrusion hole size (screen diameter) adjusted within a range from 0.5 mm to 10 mm. Thus obtained granules are dressed with, e.g., a marumerizer, dried and sieved.

The sustained release microparticle-containing granule according to the invention, which is obtained by the above method, can have an average grain diameter within the range of normally from 0.5 to 10 mm, preferably from 0.5 to 5 mm, inter alia, from 0.5 to 3 mm.

The sustained release microparticle-containing granule according to the invention can also be divided into portions and wrapped with water-soluble film, where necessary, to provide wrapped packs thereof to be thrown into paddy field, for direct application to paddy field.

### EXAMPLES

Hereinafter the present invention is explained more specifically, referring to Examples, Comparative Examples and Test Examples, it being understood that the present invention is not restricted to these Examples only. In the Examples, MAARLITE 732C (tradename, Marunaka-Hakudo Co., Ltd. average particle size: 45 μm, the floating ratio on water: 73% and water absorption: 112%) which is a foamed sirasu, was used as the microgranular form.

### Example 1

Two-hundred (200) wt % of methanol was added to 16 wt % of Pyraclonil to form a solution, into which then 64.0 wt % of microgranular form (MAARLITE 732C: tradename, Marunaka-Hakudo Co., Ltd.) was added and mixed, and the methanol was removed by vacuum distillation. To the resulting mixture, 20 wt % of a polyurethane resin (HYDRAN AP-70: tradename, DIC Corporation) was added and mixed, and then dried at 70° C. to provide sustained release microparticles.

Into a mixing vessel, 11.3 wt % of the sustained release microparticles, 0.3 wt % of alkylbenzenesulfonate (NEOPLEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 75.9 wt % of calcium carbonate (O-430: tradename, Shinko Shoji Co., Ltd.) were fed after weighing, and mixed with a universal mixer for 5 minutes. Per 100 wt % of the resulting mixture, 22 wt % of water was added, followed by further 5 minutes' kneading. The kneaded product was granulated with an extrusion granulator (dome type: Dalton Corporation) equipped with a 1.0 mmφ screen, dressed, dried and sieved (2.38-0.21 mm) to provide a sustained release microparticle-containing granule.

### Example 2

Two-hundred (200) wt % of methanol was added to 14 wt % of Pyraclonil to form a solution, into which then 56.0 wt % of microgranular form (MAARLITE 732C: tradename, Marunaka-Hakudo Co., Ltd.) was added and mixed, and the methanol was removed by vacuum distillation. To the resulting mixture, 30 wt % of a polyurethane resin (HYDRAN AP-70: tradename, DIC Corporation) was added and mixed, and then dried at 70° C. to provide sustained release microparticles.

Using 12.9 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 74.3 wt % of calcium carbonate (O-430: tradename, Shinko Shoji Co., Ltd.), a sustained release microparticle-containing granule was obtained by the method similar to Example 1.

### Example 3

Two-hundred (200) wt % of methanol was added to 12 wt % of Pyraclonil to form a solution, into which then 48.0 wt % of microgranular form (MAARLITE 732C: tradename, Marunaka-Hakudo Co., Ltd.) was added and mixed, and the methanol was removed by vacuum distillation.

To the resulting mixture, 40 wt % of a polyurethane resin (HYDRAN AP-70: tradename, DIC Corporation) was added and mixed, and then dried at 70° C. to provide sustained release microparticles.

Using 15.0 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 72.2 wt % of calcium carbonate (O-430: tradename, Shinko Shoji Co., Ltd.), a sustained release microparticle-containing granule was obtained by the method similar to Example 1.

### Example 4

Two-hundred (200) wt % of methanol was added to 16 wt % of Pyraclonil to form a solution, into which then 64.0 wt % of microgranular form (MAARLITE 732C: tradename, Marunaka-Hakudo Co., Ltd.) was added and mixed, and the methanol was removed by vacuum distillation. To the resulting mixture, 20 wt % of a polyurethane resin (HYDRAN AP-40N: tradename, DIC Corporation) was added and mixed, and then dried at 70° C. to provide sustained release microparticles.

Using 11.3 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 75.9 wt % of calcium carbonate (O-430: tradename, Shinko Shoji Co., Ltd.), a sustained release microparticle-containing granule was obtained by the method similar to Example 1.

### Example 5

Two-hundred (200) wt % of methanol was added to 14 wt % of Pyraclonil to form a solution, into which then 56.0 wt % of microgranular form (MAARLITE 732C: tradename, Marunaka-Hakudo Co., Ltd.) was added and mixed, and the methanol was removed by vacuum distillation. To the resulting mixture, 30 wt % of a polyurethane resin (HYDRAN AP-40N: tradename, DIC Corporation) was added and mixed, and then dried at 70° C. to provide sustained release microparticles.

Using 12.9 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 74.3 wt % of calcium carbonate (O-430: tradename, Shinko Shoji Co., Ltd.), a sustained release microparticle-containing granule was obtained by the method similar to Example 1.

### Example 6

Two-hundred (200) wt % of methanol was added to 12 wt % of Pyraclonil to form a solution, into which then 48.0 wt % of microgranular form (MAARLITE 732C: tradename, Marunaka-Hakudo Co., Ltd.) was added and mixed, and the methanol was removed by vacuum distillation. To the resulting mixture, 40 wt % of a polyurethane resin (HYDRAN AP-40N: tradename, DIC Corporation) was added and mixed, and then dried at 70° C. to provide sustained release microparticles.

Using 15.0 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 72.2 wt % of calcium carbonate (O-430: tradename, Shinko Shoji Co., Ltd.), a sustained release microparticle-containing granule was obtained by the method similar to Example 1.

### Example 7

Two-hundred (200) wt % of methanol was added to 14 wt % of Pyraclonil to form a solution, into which then 56.0 wt % of microgranular form (MAARLITE 732C: tradename, Marunaka-Hakudo Co. Ltd.) was added and mixed, and the methanol was removed by vacuum distillation. To the resulting mixture, 30 wt % of an acrylate resin (VONCOAT AB-901: tradename, DIC Corporation) was added and mixed, and then dried at 70° C. to provide sustained release microparticles.

Using 12.9 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 74.3 wt % of calcium carbonate (O-430: tradename, Shinko Shoji Co., Ltd.), a sustained release microparticle-containing granule was obtained by the method similar to Example 1.

### Example 8

Two-hundred (200) wt % of methanol was added to 14 wt % of Pyraclonil to form a solution, into which then 56.0 wt % of microgranular form (MAARLITE 732C: tradename, Marunaka-Hakudo Co., Ltd.) was added and mixed, and the methanol was removed by vacuum distillation. To the resulting mixture, 30 wt % of an acrylate resin (VONCOAT AB-886: tradename, DIC Corporation) was added and mixed, and then dried at 70° C. to provide sustained release microparticles.

Using 12.9 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 74.3 wt % of calcium carbonate (O-430: tradename, Shinko Shoji Co., Ltd.), a sustained release microparticle-containing granule was obtained by the method similar to Example 1.

### Example 9

Two-hundred (200) wt % of methanol was added to 14 wt % of Pyraclonil to form a solution, into which then 56.0 wt % of microgranular form (MAARLITE 732C: tradename, Marunaka-Hakudo Co. Ltd.) was added and mixed, and the methanol was removed by vacuum distillation. To the resulting mixture, 30 wt % of a butadiene-styrene copolymer resin (LACSTAR-7310-K: tradename, DIC Corporation) was added and mixed, and then dried at 70° C. to provide sustained release microparticles.

Using 12.9 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 74.3 wt % of calcium carbonate (O-430: tradename, Shinko Shoji Co., Ltd.), a sustained release microparticle-containing granule was obtained by the method similar to Example 1.

### Example 10

Two-hundred (200) wt % of methanol was added to 14 wt % of Pyraclonil to form a solution, into which then 56.0 wt % of microgranular form (MAARLITE 732C: tradename, Marunaka-Hakudo Co. Ltd.) was added and mixed, and the methanol was removed by vacuum distillation. To the resulting mixture, 30 wt % of an epoxy resin (DICFINE ENO274: tradename, DIC Corporation) was added and mixed, and then dried at 70° C. to provide sustained release microparticles.

Using, 12.9 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 74.3 wt % of calcium carbonate (O-430: tradename, Shinko Shoji Co., Ltd.), a sustained release microparticle-containing granule was obtained by the method similar to Example 1.

### Example 11

Forty (40) wt % of Pyraclonil, 5.0 wt % of polyoxyethylene allyl phenyl ether phosphate amine salt, 0.3 wt % of a silicone emulsion, 0.2 wt % of 1,2-benzoisothiazolin-3-one, and 34.5 wt % of water were mixed with THREE-ONE MOTOR (Shinto Scientific Co., Ltd.), and the solution mixture was wet-ground with DYNO-MILL (tradename, Shinmaru Enterprises Corp.) using glass beads of 1.0-1.41 mm in diameter as the grinding media. To the ground product, 15.0 wt % of 0.2% aqueous solution of xanthene gum and 5 wt % of propylene glycol were added and together homogeneously mixed with THREE-ONE MOTOR (Shinto Scientific Co., Ltd.) to provide a Pyraclonil suspension. To 35 wt % of so obtained suspension, 45.0 wt % of a microgranular form (MAARLITE 732C: tradename, Marunaka-Hakudo Co., Ltd.) was added, mixed and dried at 70° C. To the resulting mixture, further 20 wt % of a polyurethane resin (HYDRAN AP-70: tradename, DIC Corporation) was added, mixed and dried at 70° C. to provide sustained release microparticles.

Using 10.7 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 76.5 wt % of calcium carbonate (O-430: tradename, Shinko Shoji Co., Ltd.), a sustained release microparticle-containing granule was obtained by the method similar to Example 1.

### Example 12

To 30 wt % of Molinate (25° C., liquid form), 50.0 wt % of microgranular form (MAARLITE 732C: tradename, Marunaka-Hakudo Co., Ltd.) was added and mixed. To the resulting mixture, 20 wt % of a polyurethane resin (HYDRAN AP-70: tradename, DIC Corporation) was added, mixed and dried at 70° C. to provide sustained release microparticles.

Using 26.7 wt % of the Molinate-containing sustained release microparticles, 11.3 wt % of the Pyraclonil-containing sustained release microparticles as described in Example 1, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 49.2 wt % of calcium carbonate (O-430: tradename, Shinko Shoji Co., Ltd.), a sustained release microparticle-containing granule was obtained by the method similar to Example 1.

### Example 13

To 16 wt % of Pyraclonil, 64.0 wt % of microgranular form (MAARLITE 732C: tradename, Marunaka-Hakudo Co., Ltd.) and 20 wt % of a polyurethane resin (HYDRAN AP-70: tradename, DIC Corporation) were added, mixed together and dried at 70° C. to provide sustained release microparticles.

Using 11.3 wt % of the sustained release microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 75.9 wt % of calcium carbonate (O-430: tradename, Shinko Shoji Co., Ltd.), a sustained release microparticle-containing granule was obtained by the method similar to Example 1.

### Example 14

Two-hundred (200) wt % of methanol was added to 16 wt % of Pyraclonil to form a solution, into which then 64.0 wt % of microgranular form (MAARLITE 732C: tradename, Marunaka-Hakudo Co., Ltd.) was added and mixed, and the methanol was removed by vacuum distillation. To the resulting mixture, 20 wt % of a polyurethane resin (HYDRAN AP-70: tradename, DIC Corporation) was added and mixed, and then dried at 70° C. to provide sustained release microparticles.

Using 11.3 wt % of so obtained sustained release microparticles, 2.5 wt % of a sodium salt of carboxymethyl cellulose (CELLOGEN 7A: tradename, Dai-ichi Kogyo Seiyaku Co.) in place of polyvinyl alcohol, and 75.9 wt % of calcium carbonate (O-430: tradename, Shinko Shoji Co., Ltd.), a sustained release microparticle-containing granule was obtained by the method similar to Example 1.

### Example 15

After adding 25.5 wt % of microgranular form (MAARLITE 732C: tradename, Marunaka-Hakudo Co., Ltd.) to 59.5 wt % of Pyraclonil and mixing them, 15 wt % of a polyurethane resin (SUPERFLEX 420: tradename, Dai-ichi Kogyo Seiyaku Co.) was added to the resulting mixture, mixed together, and dried at 70° C. to provide sustained release microparticles.

Using 3.36 wt % of the sustained release microparticles, 0.8 wt % of sodium polycarboxylate (SHALLOLAN103P: tradename, Dai-ichi Kogyo Seiyaku Co.), 0.35 wt % of dialkylsulfosuccinate sodium salt (NEOCOL YSK: tradename, Dai-ichi Kogyo Seiyaku Co.), 30 wt % of bentonite (KUNIGEL-V1: tradename, Kunimine Industries Co., Ltd.), and 65.49 wt % of calcium carbonate (O-430: tradename, Shinko Shoji Co., Ltd.), a sustained release microparticle-containing granule was obtained by the method similar to Example 1.

### Example 16

After adding 34.0 wt % of microgranular form (MAARLITE 732C: tradename, Marunaka-Hakudo Co., Ltd.) to 51.0 wt % of Pyraclonil and mixing them, 15 wt % of a polyurethane resin (SUPERFLEX 420: tradename, Dai-ichi Kogyo Seiyaku Co.) was added to the resulting mixture, mixed together, and dried at 70° C. to provide sustained release microparticles.

Using 3.92 wt % of the sustained release microparticles, 0.8 wt % of sodium polycarboxylate (SHALLOLAN103P: tradename, Dai-ichi Kogyo Seiyaku Co.), 0.35 wt % of dialkylsulfosuccinate sodium salt (NEOCOL YSK: tradename, Dai-ichi Kogyo Seiyaku Co.), 30 wt % of bentonite (KUNIGEL-V1: tradename, Kunimine Industries Co., Ltd.) and 64.93 wt % of calcium carbonate (O-430: tradename, Shinko Shoji Co., Ltd.), a sustained release microparticle-containing granule was obtained by the method similar to Example 1.

### Example 17

After adding 42.5 wt % of microgranular form (MAARLITE 732C: tradename, Marunaka-Hakudo Co., Ltd.) to 42.5 wt % of Pyraclonil and mixing them, 15 wt % of a polyurethane resin (SUPERFLEX 420: tradename, Dai-ichi Kogyo Seiyaku Co.) was added to the resulting mixture, mixed together, and dried at 70° C. to provide sustained release microparticles.

Using 4.71 wt % of the sustained release microparticles, 0.8 wt % of sodium polycarboxylate (SHALLOLAN103P; tradename, Dai-ichi Kogyo Seiyaku Co.), 0.35 wt % of dialkylsulfosuccinate sodium salt (NEOCOL YSK: tradename, Dai-ichi Kogyo Seiyaku Co.), 30 wt % of bentonite (KUNIGEL-V1; tradename, Kunimine Industries Co., Ltd.) and 64.14 wt % of calcium carbonate (O-430: tradename, Shinko Shoji Co., Ltd.), a sustained release microparticle-containing granule was obtained by the method similar to Example 1.

### Example 18

After adding 59.5 wt % of Pyraclonil to 25.5 wt % of microgranular form (MAARLITE 732C: tradename, Marunaka-Hakudo Co., Ltd.) and mixing them, 15 wt % of a polyurethane resin (SUPERFLEX 650; tradename, Dai-ichi Kogyo Seiyaku Co.) was added to the resulting mixture, mixed together, and dried at 70° C. to provide sustained release microparticles.

Using 3.36 wt % of the sustained release microparticles, 0.8 wt % of sodium polycarboxylate (SHALLOLAN103P; tradename, Dai-ichi Kogyo Seiyaku Co.), 0.35 wt % of dialkylsulfosuccinate sodium salt (NEOCOL YSK: tradename, Dai-ichi Kogyo Seiyaku Co.), 30 wt % of bentonite (KUNIGEL-V1: tradename, Kunimine Industries Co., Ltd.) and 65.49 wt % of calcium carbonate (O-430: tradename, Shinko Shoji Co., Ltd.), a sustained release microparticle-containing granule was obtained by the method similar to Example 1.

### Example 19

Pyraclonil in the amount of 47.6 wt % was homogeneously mixed with 11.9 wt % of calcium carbonate (O-430: tradename, Shinko Shoji Co., Ltd.), and to the mixture 25.5 wt % of hollow microspheres (MAARLITE 732C: tradename, Marunaka-Hakudo Co., Ltd.) was added and mixed. To the resulting mixture, 15 wt % of a polyurethane resin (SUPERFLEX 420: tradename, Dai-ichi Kogyo Seiyaku Co.) was added, mixed, and dried at 70° C. to provide sustained release microparticles.

Using 4.20 wt % of the sustained release microparticles, 0.8 wt % of sodium polycarboxylate (SHALLOLAN103P; tradename, Dai-ichi Kogyo Seiyaku Co.), 0.35 wt % of dialkylsulfosuccinate sodium salt (NEOCOL YSK: tradename, Dai-ichi Kogyo Seiyaku Co.), 30 wt % of bentonite (KUNIGEL-V1: tradename, Kunimine Industries Co., Ltd.) and 64.65 wt % of calcium carbonate (O-430: tradename, Shinko Shoji Co., Ltd.), a sustained release microparticle-containing granule was obtained by the method similar to Example 1.

### Comparative Example 1

Two-hundred (200) wt % of methanol was added to 16 wt % of Pyraclonil to form a solution, into which 84.0 wt % of microgranular form (MAARLITE 732C: tradename, Marunaka-Hakudo Co. Ltd.) was added and mixed, and the methanol was removed by vacuum distillation to provide comparative microparticles.

Using 11.3 wt % of the comparative microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 75.9 wt % of calcium carbonate (O-430: tradename, Shinko Shoji Co., Ltd.), a comparative granule was obtained by the method similar to Example 1.

### Comparative Example 2

Two-hundred and eighty (280) wt % of methanol was added to 16 wt % of Pyraclonil to form a solution, to which 64.0 wt % of porous silica (CARPLEX #80: tradename, Evonik Degussa Japan Co., Ltd.) having an average particle size of 8.1 μm, floating ratio on water of 0% and water absorption of 250% was added and mixed. Thereafter the methanol was removed by vacuum distillation. To the remaining mixture, 20 wt % of a polyurethane resin (HYDRAN AP-70: tradename, DIC Corporation) was added, mixed and dried at 70° C. to provide comparative microparticles.

Using 11.3 wt % of the comparative microparticles, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 75.9 wt % of calcium carbonate (O-430: tradename, Shinko Shoji Co., Ltd.), a comparative granule was obtained by the method similar to Example 1.

### Comparative Example 3

Into a mixing vessel, 1.8 wt % of Pyraclonil, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 85.4 wt % of calcium carbonate (O-430: tradename, Shinko Shoji Co., Ltd.) were fed after weighing and mixed with a universal mixer for 5 minutes. Per 100 wt % of the resulting mixture, 15 wt % of water was added, followed by further 5 minutes' kneading. The kneaded product was granulated with an extrusion granulator (dome type: Dalton Corporation) equipped with a 1.0 mmφ screen, dressed, dried and sieved (2.38-0.21 mm) to provide a comparative granule.

### Comparative Example 4

Into a mixing vessel, 0.3 wt % of alkylbenzenesulfonate (NEOPELEX No. 6F: tradename, KAO Corporation), 2.5 wt % of polyvinyl alcohol (GOHSENOL GL-05S: tradename, The Nippon Synthetic Chemical Industry Co., Ltd.), 10.0 wt % of bentonite (KUNIGEL-V2: tradename, Kunimine Industries Co., Ltd.) and 79.2 wt % of calcium carbonate (O-430: tradename, Shinko Shoji Co., Ltd.) were fed after weighing and mixed with a universal mixer for 5 minutes. Per 92 wt % of the resulting mixture, 15 wt % of water was added, followed by further 5 minutes' kneading. The kneaded product was granulated with an extrusion granulator (dome type: Dalton Corporation) equipped with a 1.0 mmφ screen, dressed, dried and sieved (2.38-0.21 mm) to provide a granule. A comparative granule was obtained by adding 8 wt % of Molinate bulk to 92 wt % of the granule to have it absorb the oil form of Molinate.

### Test Example 1

Sustained release microparticles as obtained in Examples 1, 4, 11, 13 and 15-18, and the comparative microparticles of Comparative Examples 1 and 2 were given the following test.

### [Elution-in-Water Test of the Sustained Release Microparticles]

Into 500 mL-beakers each containing 250 mL of 20° C. of 3° hard water, 20 mg each of the sustained release microparticles or the comparative microparticles as prepared in the above were scattered. Twenty-four (24) hours thereafter, from the central part of each beaker 1 mL of the water therein was collected. Thereafter the agrochemically active component was thoroughly dispersed in the water in each beaker with an ultrasonic generator, and again 1 ml each of the aqueous samples were collected. The concentration of the agrochemically active component in these samples was measured by means of liquid chromatography, and the elution-in-water was calculated from the measured value, according to the following equation. The results were as given in Table 1.

Elution-in-water(%)=(Ct/Cx)×100
Ct: agrochemically active component's concentration after 24 hours of the scattering.
Cx: agrochemically active component's concentration after thorough dispersion of the component by ultrasonic waves applied to the beakers inclusive of their contents, with an ultrasonic generator (AIWA Medical Industry Co., Ltd., oscillation output: 220W).

When the microparticles tested were forcibly dispersed for 3 minutes with the above ultrasonic generator, their elution-in-water was invariably 100%.

TABLE 1

| Sustained Release | | Elution-in-water (%) |
|---|---|---|
| microparticles | Component | 24 hrs. |
| Example 1 | Pyraclonil | 26.1 |
| Example 4 | Pyraclonil | 22.5 |
| Example 11 | Pyraclonil | 31.2 |
| Example 13 | Pyraclonil | 32.8 |
| Example 15 | Pyraclonil | 20.1 |
| Example 16 | Pyraclonil | 32.5 |
| Example 17 | Pyraclonil | 45.2 |
| Example 18 | Pyraclonil | 38.8 |
| Comparative Example 1 | Pyraclonil | 75.3 |
| Comparative Example 2 | Pyraclonil | 70.4 |

As shown in above Table 1, those sustained release microparticles which were prepared in Examples 1, 4, 11, 13 and 15-18 were found to excel in the ability to control the elution rate of the agrochemically active component into water, as compared to those of the comparative microparticles of Comparative Examples 1 and 2.

### Test Example 2

Sustained release microparticle-containing granules of Examples 1-19 and the comparative granules of Comparative Examples 1-4 were given the following test.

### [Elution-In-Water Test of Sustained Release Microparticle-Containing Granules]

The elution-in-water of sustained release microparticle-containing granules was determined by the method similar to that used in Test Example 1. The sampling was done at 24 and 72 hours of the scattering. The sampling was also done at 72 hours after the microparticle-containing granules were thoroughly dispersed with the ultrasonic generator. The results were as shown in Table 2.

When the microparticle-containing granules were forcibly dispersed for 3 minutes with the ultrasonic generator, their elution-in-water was invariably 100%.

TABLE 2

| Sustained Release | | | |
|---|---|---|---|
| Microparticle— | | Elution-in-Water (%) | |
| containing Granule | Component | 24 hrs. | 72 hrs. |
| Example 1 | Pyraclonil | 38.7 | 58.7 |
| Example 2 | Pyraclonil | 34.6 | 52.3 |
| Example 3 | Pyraclonil | 15.9 | 36.3 |
| Example 4 | Pyraclonil | 34.9 | 52.3 |
| Example 5 | Pyraclonil | 30.4 | 48.5 |
| Example 6 | Pyraclonil | 19.5 | 37.8 |
| Example 7 | Pyraclonil | 44.4 | 69.4 |
| Example 8 | Pyraclonil | 45.8 | 70.7 |
| Example 9 | Pyraclonil | 63.2 | 83.5 |
| Example 10 | Pyraclonil | 53.6 | 78.4 |
| Example 11 | Pyraclonil | 43.6 | 65.7 |
| Example 12 | Pyraclonil | 36.5 | 56.4 |
| | Molinate | 55.5 | 81.4 |
| Example 13 | Pyraclonil | 43.6 | 65.7 |
| Example 14 | Pyraclonil | 30.5 | 55.2 |
| Example 15 | Pyraclonil | 20.5 | 35.8 |
| Example 16 | Pyraclonil | 45.4 | 70.4 |
| Example 17 | Pyraclonil | 62.8 | 81.7 |
| Example 18 | Pyraclonil | 47.2 | 68.5 |
| Example 19 | Pyraclonil | 20.2 | 35.7 |
| Comparative Example 1 | Pyraclonil | 85.0 | 100 |
| Comparative Example 2 | Pyraclonil | 78.0 | 100 |
| Comparative Example 3 | Pyraclonil | 100 | 100 |
| Comparative Example 4 | Molinate | 100 | 100 |

As shown in Table 2, those sustained release microparticle-containing granules according to the present invention, which were prepared in Examples 1-19, were found to excel in the ability to control the elution rate of the agrochemically active component into water, as compared to the comparative granules of Comparative Examples 1-4.

1-21. (canceled)

22. Sustained release microparticles comprising a supported substance, microgranular form having a hollow structure and coating agent which is at least one member selected from the group consisting of thermosetting resins, characterized in that the microgranular form has an average particle size within a range from 20 to 300 μm.

23. Sustained release microparticles according to claim 22, which contain, based on the weight of the sustained release microparticles, from 0.1 to 95 wt % of the supported substance, from 0.1 to 90 wt % of the microgranular form, and from 0.1 to 60 wt % of the coating agent.

24. Sustained release microparticles according to claim 22, in which the microgranular form has a floating ratio on water of from 20 to 95% and water absorption of from 10 to 170%.

25. Sustained release microparticles according to claim 22, in which the microgranular form is of hollow glass microspheres.

26. Sustained release microparticles according to claim 22, in which the microgranular form is at least one member selected from the group consisting of foamed pearlite, foamed glass, fly ash and hollow microballoon.

27. Sustained release microparticles according to claim 22, in which the microgranular form has an average particle size within a range from 20 to 150 μm.

28. Sustained release microparticles according to claim 22, in which the microgranular form has an average particle size within a range from 20 to 80 μm.

29. Sustained release microparticles according to claim 22, in which the supported substance is at least one member selected from the group consisting of agrochemically active component, coloring matter, flavor and fragrance, functional substance, medicament, fertilizer component, enzyme and physiologically active substance.

30. Sustained release microparticles according to claim 29, in which the supported substance is an agrochemically active component.

31. Sustained release microparticles according to claim 22, in which the coating agent is a thermosetting polyurethane resin.

32. A sustained release microparticle-containing preparation which comprises at least one kind of the sustained release microparticles according to claim 22.

33. A sustained release microparticle-containing preparation according to claim 32, which is in the form of granule, fine granule, tablet, wettable powder, water dispersible granule, dustable powder, suspension concentrate, emulsion water in oil, emulsion oil in water, soluble concentrate, emulsifiable concentrate, or oil miscible liquid.

34. A sustained release microparticle-containing preparation according to claim 32, which is used as at least one member selected from the group consisting of agrochemical, fertilizer, coating agent, detergent, aromatic, catalyst, controlling agent and antibacterial agent.

35. A sustained release microparticle-containing preparation according to claim 33, in which the granule has a grain diameter ranging from 0.5 mm to 10 mm.

36. A granule according to claim 35, which comprises at least one kind of sustained release microparticles comprising a supported substance, microgranular form having a hollow structure and coating agent which is at least one member selected from the group consisting of thermosetting resins, characterized in that the microgranular form has an average particle size within a range from 20 to 300 μm, surfactant, binder and carrier.

37. A method for preparing the sustained release microparticles as described in claim 22, comprising mixing the substance to be supported, microgranular form and coating agent, and drying the mixture.

38. A method for preparing the sustained release microparticles as described in claim 22, comprising either mixing the substance to be supported with the microgranular form, further adding thereto a coating agent, mixing and drying the mixture; or mixing the microgranular form with a coating agent, further adding thereto the substance to be supported, mixing and drying the mixture; or mixing the substance to be supported with a coating agent, further adding thereto the microgranular form, mixing and drying the mixture.

39. A method for preparing the sustained release microparticles as described in claim 22, comprising dissolving the substance to be supported in an organic solvent, mixing the formed solution with the microgranular form, distilling the organic solvent off, and thereafter mixing a coating agent with the residue, and drying the mixture; or dissolving the substance to be supported in an organic solvent, mixing the solution with the microgranular form and a coating agent, distilling the organic solvent off, and thereafter further drying the coating agent.

40. A method for preparing the sustained release microparticles as described in claim 22, comprising dissolving or dispersing the substance to be supported in water and a water-soluble medium, mixing the solution or dispersion with the microgranular form, drying the same, mixing it further with a coating agent and drying the mixture; or dissolving or dispersing the substance to be supported in water and a water-soluble medium, mixing the resulting solution or dispersion with the microgranular form and a coating agent, and drying the mixture.
